(19) United States
(12) Patent Application Publication
(10) Pub. No.: US 2025/0146988 A1
(43) Pub. Date: May 8, 2025

(54) METHOD AND MARKERS FOR IDENTIFYING COLD SENSITIVITY OF DIFFERENT PEACH VARIETIES

(71) Applicant: Zhejiang Wanli University, Ningbo (CN)

(72) Inventor: Chunbo Song, Ningbo (CN)

(21) Appl. No.: 18/654,008

(22) Filed: May 3, 2024

(30) Foreign Application Priority Data
Nov. 6, 2023 (CN) 2023114615797

(51) Int. Cl. G01N 30/88; G01N 30/02; G01N 33/02

(52) U.S. Cl. CPC G01N 30/88; G01N 33/025; G01N 2030/027

(57) ABSTRACT

A method and markers for identifying cold sensitivity of different peach varieties, which relates to the field of fruit preservation technologies. According to lipid metabolomics identification and big data analysis, compositions of sphingolipids in different peach varieties are extracted, common components of sphingolipids of different varieties are compared, so as to provide a method for identifying a CI process of different peach varieties based on sphingolipid metabolism.

### CROSS-REFERENCE TO RELATED APPLICATION

This application claims priority to Chinese Patent Application No. 202311461579.7, filed Nov. 6, 2023, which is herein incorporated by reference in its entirety.

### TECHNICAL FIELD

The disclosure relates to the field of fruit preservation technologies, and more particularly to a method and markers for identifying cold sensitivity of different peach varieties.

### BACKGROUND

Peach (also referred to as Prunus persica (L.) Batsch) has juicy flesh and rich nutrition, but it is prone to chilling injury (CI) when stored at temperature lower than 10° C. The main symptoms of CI include flesh browning, flesh mealiness, flesh lignification, flesh abnormal maturity and flesh perishable, which seriously affects edible value and commodity value of the peach, resulting in postharvest quality deterioration and corruption of peach fruit. Therefore, it has an important practical significance to research the occurrence and control mechanism of CI in peach fruits after harvesting, so as to provide a theoretical basis to improve quality and overall competitiveness of the fruit and develop postharvest storage and preservation technologies.

Sphingolipids, as an important structural component lipid of plasma membrane and other inner membrane systems (also referred to as endomembrane systems), are widely present in eukaryotes and some prokaryotic membranes. Steady-state balance of components of the sphingolipids plays an important role in maintaining stability and fluidity of the membrane system. The sphingolipids are a kind of complex compound with sphingosine as a backbone, and are divided into ceramide (CER), sphingomyelin (SM) and hexosylceramide (HexCer) according to complexity and diversity of polar head bases. At present, there are few reports on a mechanism of CI of fruits mediated by sphingolipid metabolism, and there is no method to identify the CI process of different peach varieties based on the sphingolipid metabolism.

### SUMMARY

In order to solve the above problems, the disclosure provides markers for identifying cold sensitivity of different peach varieties, and the markers includes: CER, SM and glycosphingolipid (i.e., hexosylceramide, HexCer).

The disclosure further provides an application method of the above markers, and the application method includes:
identifying the cold sensitivity of different peach varieties by using the markers.

The disclosure further provides a method for identifying cold sensitivity of different peach varieties, and the method includes:
determining the cold sensitivity of different peach varieties through comparing changes in fold change of CER, SM and HexCer in different peach varieties at low-temperature storage with at harvest.

In an embodiment, the method further includes:
detecting the CER, the SM and the HexCer as markers for identifying the cold sensitivity of different peach varieties by using a SCIEX ExionLC™ ultra-high performance liquid chromatograph.

In an embodiment, the CER and the HexCer increase, indicated by the fold change of the CER and the HexCer greater than 1, and the SM decreases, indicated by the fold change of the SM smaller than 1, indicating differences in cold sensitivity among different peach varieties. The disclosure has the following beneficial effects.

The disclosure is a method for identifying CI process of different peach varieties based on sphingolipid metabolism. The method is simple and easy to operate, and can mechanically process a large number of samples. At the same time, the obtained data excludes influence of objective factors such as human factors. Through the method, data processing can achieve stereotype and identify the CI process of different peach varieties according to corresponding standards. The method is innovative and provides a determination basis for the identification of the cold-sensitive peach varieties and the optimization of peach fruit CI control technology.

### DETAILED DESCRIPTION OF EMBODIMENTS

Multiple exemplary embodiments of the disclosure are described in detail. In the embodiments, conventional methods are used unless otherwise specified, and reagents used are either commercially available or configured using conventional methods unless otherwise specified. This detailed description should not be considered as a limitation of the disclosure, but should be understood as a more detailed description of certain aspects, features, and embodiments of the disclosure.

It should be understood that terms described in the disclosure are merely for describing specific embodiments and are not intended to limit the disclosure. Furthermore, for a numerical range in the disclosure, it should be understood that each intermediate value between upper and lower limits of the range is also specifically disclosed. Each smaller range between any stated value or intermediate value within the range, as well as any other stated value or intermediate value within the range, is also included in the disclosure. The upper and lower limits of these smaller ranges can be independently included or excluded within the range.

Unless otherwise specified, all technical and scientific terms used in this article have the same meanings as those commonly understood by those skilled in the art described herein. Although the disclosure only describes some methods and materials, any methods and materials similar or equivalent to those described herein may also be used in the implementation or testing of the disclosure. All literature mentioned in the specification is incorporated by reference to publicly disclose and describe methods and/or materials related to the literature. In case of conflict with any incorporated literature, the content of the specification shall prevail.

It is evident to those skilled in the art that various improvements and variations can be made to the specific embodiments of the disclosure without departing from a scope or spirit of the disclosure. The other embodiments obtained from the specification of the disclosure are obvious to those skilled in the art. The specification and embodiments of the disclosure are only illustrative.

Terms “including”, “comprising”, “having”, “containing” and the like used in this article are all open-ended terms, meaning to include but not limited to.

### Embodiment

1. Different varieties of peach fruit (includes Chibaifen, Qiongjiangyulu, liangfang, Baili and Hujing, which can be purchased from Ningbo city, Zhejiang province) are stored at an incubator with a temperature of 4° C., and peach flesh samples of different varieties respectively stored for 0 day (d) and 21 d are taken to extract and determine components of sphingolipids. Fold change represents multiple of accumulation of the sphingolipids during low-temperature storage for 21 d relative to harvest (i.e., 0 d). When the fold change is greater than 1, indicating that low-temperature induced increase in sphingolipid components, and the greater the fold change, the more the low-temperature induced increase in the sphingolipid components. When the fold change is less than 1, indicating that low-temperature induced decrease in the sphingolipid components, and the less the fold change, the more the low-temperature induced decrease in the sphingolipid components.

### 2. Determination of Lipidomics in Peach Fruit

### 2.1 Extraction of Metabolites

100 milligrams (mg) of each sample are weighed on dry ice, steel balls and 400 microliters (μL) of water are added into the sample, and the sample added with steel balls and water is mixed evenly by vortex for 60 seconds(s) to obtain a mixture. The mixture is ground for 4 minutes (min) and is ultrasonically treated for 5 min (ice water bath) at 45 hertz (Hz), and the above steps are repeated for three times to obtain a homogenate. 960 μL of extraction solution (methyl tert-butyl ether abbreviated as MTBE:methanol abbreviated as MeOH is 5:1) is added into 400 μL of the homogenate to obtain a mixed solution. The mixed solution is mixed evenly by vortex for 60 s, and is ultrasonically treated at ice water bath for 10 min to obtain a first sample. The first sample is centrifuged at 4° C. and 3000 revolutions per minute (rpm) for 15 min to obtain a first supernatant. 500 μL of MTBE is added into 500 μL of the first supernatant, and the first supernatant added with MTBE is mixed evenly by vortex for 60 s and ultrasonically treat for 10 min to obtain a second sample. The second sample is centrifuged at 4° C. and 3000 rpm for 15 min to obtain a second supernatant. 500 μL of MTBE is added into 500 μL of the second supernatant, and the second supernatant added with the MTBE is mixed evenly by vortex for 60 s and ultrasonically treat for 10 min to obtain a third sample. The third sample is centrifuged at 4° C. and 3000 rpm for 15 min to obtain a third supernatant. The above three supernatants are merged and vacuum dried at 37° C. to obtain dried metabolites. The dried metabolites are dissolved in 100 μL of solution (dichloromethane abbreviated as DCM:MeOH: water abbreviated as H₂O=60:30:4.5) to be mixed evenly by vortex for 60 s and ultrasonically treated at ice water bath for 10 min to obtain a fourth sample. The fourth sample is centrifuged at 4° C. and 12000 rpm for 15 min to obtain a fourth supernatant. 30 μL of the fourth supernatant is added into a sample vial (also referred to as sample bottle) for machine determination.

### 2.2 Machine Determination

A SCIEX ExionLC™ ultra-high performance liquid chromatograph is used for chromatographic separation of the markers. A phase A of the liquid chromatograph is 40% water and 60% acetonitrile solution, which includes 10 millimoles per liter (mmol/L) ammonium acetate. A phase B of the liquid chromatograph is 10% acetonitrile and 90% isopropanol solution, which includes 10 mmol/L ammonium acetate. A flow rate of a mobile phase is 0.3 milliliters per minute (mL/min), a column temperature is 40° C., a sample disk temperature is 6° C., and an injection volume is 2 μL.

Parameters of an AB Sciex QTrap™ 6500 mass spectrometer are set as follows. When collecting data, a multiple reaction monitoring (MRM) mode is used to perform mass spectrometry analysis. Ion source parameters are as follows: ion spray voltage: +5500/−4500 volts (V), curtain gas: 40 pounds per square inch (psi), temperature: 350° C., ion source gas 1:50 psi, ion source gas 2:50 psi, DP: ±80 V.

### 3. Data Process of Lipidomics

Data preprocessing and annotation of Liquid chromatography-mass spectrometry/mass spectrometry (LC-MS/MS) analysis include the following steps. A “msconvert” program of “Proteo Wizard” is used to convert raw data document to a document with a mzXML form. A peak detection should be used for primary mass spectrometry (MS1) data. A CentWave algorithm of XCMS is used to use a MS/MS spectrum for peak detection, and lipid identification is achieved by using a spectral matching of a LipidBlast base.

### 4. Identification of CI Process in Different Peach Varieties Based on Sphingolipid Metabolism

Results analysis are as follows. Sphingolipid components accumulated by different peach varieties at low temperature include Cer, HexCer and SM. Low temperature induced an increase in the components of Cer and HexCer, the fold changes of which are greater than 1 and higher than that of other varieties, while the component of SM decreased, the fold change of which is less than 1 and lower than that of other varieties, indicating that this variety has significant CI. The data are shown in Table 1.

TABLE 1
Testing data results

| | Fold Change | | | | |
|---|---|---|---|---|---|
| Lipid Class | Chibaifen | Qiongjiangyulu | Liangfang | Baili | Hujing |
| Cer-ADS d17:0/19:1 | 366.8676 | 11.9706 | 75.4623 | 24.8856 | 27.0796 |
| Cer-AP t16:1/18:0 | 293.7826 | 26.0790 | 105.1247 | 150.6829 | 124.0084 |
| Cer-AP t18:0/26:0 | 26.5224 | 3.2057 | 5.7332 | 7.3646 | 7.2569 |
| Cer-AP t18:0/27:0 | 46.2209 | 4.0628 | 4.5518 | 10.2737 | 6.7231 |
| Cer-AP t18:1/21:0 | 142.1462 | 10.5751 | 36.7446 | 27.1708 | 23.2951 |
| Cer-AP t18:1/23:0 | 20.1796 | 3.2773 | 7.3227 | 6.2111 | 5.2683 |
| Cer-AP t18:1/26:0 | 21.2463 | 3.9072 | 6.0369 | 7.5156 | 5.1297 |
| Cer-AP t20:0/24:1 | 23.3399 | 3.9678 | 6.9942 | 7.0185 | 5.3581 |
| Cer-AP t22:0/19:0 | 19.5669 | 2.5956 | 3.1614 | 4.1664 | 4.2450 |
| Cer-AP t22:1/18:0 | 38.1745 | 4.9452 | 13.0771 | 9.1896 | 9.3571 |
| Cer-NP t18:1/16:0 | 21.1017 | 12.7096 | 24.9738 | 13.3158 | 16.3471 |
| HexCer-AP | 2.4615 | 3.8860 | 5.5848 | 6.5674 | 6.5734 |
| t22:1/20:1 | | | | | |
| HexCer-NS | 6.9094 | 4.7366 | 3.9420 | 2.2365 | 3.9988 |
| d14:3/20:2 | | | | | |
| SM d14:1/23:1 | 0.0668 | 0.5016 | 0.3576 | 0.2524 | 0.4770 |

What is claimed is:

1. Markers for identifying cold sensitivity of different peach varieties, comprising: ceramide (CER), sphingomyelin (SM) and hexosylceramide (HexCer).

2. An application method of the markers as claimed in claim 1, comprising:
identifying the cold sensitivity of different peach varieties by using the markers.

3. A method for identifying cold sensitivity of different peach varieties, comprising:
determining the cold sensitivity of different peach varieties through comparing changes in fold change of CER, SM and HexCer in different peach varieties at low-temperature storage with at harvest.

4. The method as claimed in claim 3, wherein the method further comprises:
detecting the CER, the SM and the HexCer as markers for identifying the cold sensitivity of different peach varieties by using a SCIEX ExionLC™ ultra-high performance liquid chromatograph.

5. The method as claimed in claim 3, wherein the CER and the HexCer increase, indicated by the fold change of the CER and the HexCer greater than 1, and the SM decreases, indicated by the fold change of the SM smaller than 1, indicating differences in cold sensitivity among different peach varieties.

6. The method as claimed in claim 3, wherein the determining cold sensitivity of different peach varieties through comparing changes in fold change of CER, SM and HexCer in different peach varieties at low-temperature storage with at harvest specifically comprises:
determining the cold sensitivity of different peach varieties through comparing the changes in the fold change of CER, SM and HexCer in the different peach varieties at low-temperature storage lower than 10 Celsius degrees (° C.) with at harvest.
